(19) United States
(12) Patent Application Publication
(10) Pub. No.: US 2026/0212994 A1
(43) Pub. Date: Jul. 23, 2026

(54) SYSTEM AND METHOD FOR INTEGRATED FITNESS TRACKING WITH HOLISTIC HEALTH MANAGEMENT AND AI-ENHANCED USER ENGAGEMENT

(71) Applicants: LEON NEIL CODNER, RIVERVIEW, FL (US); TYCELIA CODNER, RIVERVIEW, FL (US)

(72) Inventors: LEON NEIL CODNER, RIVERVIEW, FL (US); TYCELIA CODNER, RIVERVIEW, FL (US)

(21) Appl. No.: 19/031,949

(22) Filed: Jan. 18, 2025

(51) Int. Cl. G16H 20/90; A63B 24/00; G06Q 30/0214; G06Q 30/0251; G06Q 30/0601; G06Q 50/00; G16H 10/60; G16H 20/30; G16H 40/67

(52) U.S. Cl. CPC G16H 20/90; A63B 24/0059; A63B 24/0062; A63B 24/0075; G06Q 10/40; G06Q 30/0214; G06Q 30/0261; G06Q 30/0267; G06Q 30/0603; G16H 10/60; G16H 20/30; G16H 40/67; A63B 2024/0065; A63B 2225/20

(57) ABSTRACT

A computing system and method for integrated fitness tracking and holistic health management is disclosed. The system comprises subsystems for customizable user experience, holistic health management, AI-driven personalization, integrated marketing capabilities, wearable device integration, sustainable lifestyle promotion, marketing and community engagement, e-commerce, beacon technology, personalized meal suggestions, smart fabrics, AI model interoperability, contact and data sharing, and AI voice interaction with real-time feedback. The system utilizes user inputs, biometric data, wearable device data, and artificial intelligence to provide personalized workout plans, nutritional guidance, and targeted marketing. The system also promotes sustainable practices, facilitates community engagement, and leverages beacon technology to enhance user experience in retail locations. This comprehensive approach optimizes user health, fitness, and overall well-being while offering a personalized and engaging experience.

### TECHNICAL FIELD

The present invention relates generally to the field of mobile health and fitness technology. More specifically, it pertains to a system and method configured to provide a comprehensive, personalized, and sustainable approach to fitness and wellness through features such as customizable workout plans, integrated health tracking, AI-driven insights, social community engagement, and targeted marketing strategies.

### BACKGROUND OF THE INVENTION

The field of mobile health and fitness technology has witnessed significant growth in recent years, driven by the increasing popularity of smartphones and wearable devices, coupled with a growing awareness of the importance of health and wellness. Numerous fitness applications have emerged, offering a variety of features such as activity tracking, workout routines, and nutrition guidance.

However, existing fitness applications often suffer from several limitations. Many apps provide generic workout plans that may not be tailored to individual needs and preferences, leading to suboptimal results and decreased motivation. Additionally, these apps often focus solely on physical fitness, neglecting other crucial aspects of well-being such as nutrition, sleep, and mental health.

Furthermore, many fitness applications lack effective mechanisms for long-term user engagement. Without personalized guidance, social support, and gamification elements, users may quickly lose interest and abandon their fitness goals. Existing apps also frequently fail to leverage the vast amounts of user data they collect, missing opportunities for data-driven insights that could enhance user experience and personalization.

Another significant drawback of current fitness applications is their limited integration with other aspects of a user's life. For instance, many apps do not seamlessly connect with wearable devices, resulting in fragmented data and a less comprehensive view of a user's health. Moreover, there is a lack of integration with social media platforms, hindering users from sharing their progress and receiving support from their network.

Finally, few fitness applications prioritize sustainability or consider the environmental impact of their operations. With the increasing awareness of climate change and the need for eco-conscious practices, there is a growing demand for sustainable solutions in all aspects of life, including fitness technology.

In light of these limitations, there exists a need for a comprehensive fitness application that addresses the aforementioned drawbacks. Such an application should offer personalized workout plans, integrated health tracking, AI-driven insights, social community engagement, targeted marketing strategies, and a commitment to sustainability. The present invention aims to fulfill this need by providing a novel and advanced solution that revolutionizes the way users approach fitness and wellness.

### SUMMARY OF THE INVENTION

The present invention addresses the aforementioned shortcomings by providing a system and method for integrated customized fitness tracking with holistic health management and AI-enhanced user engagement.

A computing system for integrated fitness tracking and holistic health management is disclosed. The computing system comprises of a a hardware processor and a memory coupled to the hardware processor, wherein the memory comprises a set of program instructions in the form of a plurality of subsystems, configured to be executed by the hardware processor. The plurality of subsystems comprises a customizable user experience subsystem configured to receive user inputs regarding preferred activities and audio feedback, integrate with external music streaming services, and dynamically adjust a user interface based on user preferences.

The plurality of subsystems also comprises a holistic health management subsystem configured to track and record fasting periods, physical activities, hydration, and sleep patterns, and provide rewards and incentives to users based on their health and fitness progress.

The plurality of subsystems also comprises an AI-driven personalization subsystem configured to collect and analyze user-specific data, generate personalized workout plans and recommendations, and configured to adapt plans based on changing user needs and progress.

The plurality of subsystems also comprises an integrated marketing capabilities subsystem configured to integrate with social media platforms to share user milestones, administer referral incentives and loyalty programs, and deliver targeted advertisements and promotions for fitness-related products and services.

The plurality of subsystems also comprises a wearable device integration subsystem configured to synchronize real-time data from wearable devices and analyze the data to provide detailed insights into fitness metrics.

The plurality of subsystems also comprises a sustainable lifestyle promotion subsystem configured to minimize the environmental footprint of the system, encourage users to adopt eco-friendly habits, and enhance the energy efficiency of the system's operation.

The plurality of subsystems also comprises a marketing and community engagement subsystem configured to use geofencing to deliver targeted messages based on location, facilitate community interaction and competition, and employ precision marketing tools to target specific user demographics.

The plurality of subsystems also comprises an E-commerce subsystem configured to present a catalog of food, beverages, and fitness products, provide personalized product recommendations, facilitate secure shopping, checkout, and order management, and offer promotions, discounts, and subscription services.

The plurality of subsystems also comprises a beacon subsystem configured to manage physical beacons in retail locations, detect user proximity to beacons, integrate with user data and AI to deliver personalized promotions and navigation, and provide retailers with data analytics for optimizing in-store strategies.

The plurality of subsystems also comprises a personalized meal suggestion subsystem configured to acquire biometric data from wearable sensors, analyze and interpret biometric data, and generate personalized meal and hydration recommendations based on user data and biometric analysis.

The plurality of subsystems also comprises a smart fabrics subsystem configured to incorporate sensors in wearable garments to collect biometric and biomechanical data, transmit data wirelessly to the computing system, and provide real-time feedback, personalized workout recommendations, and progress tracking.

The plurality of subsystems also comprises an AI model interoperability subsystem configured to facilitate communication and data exchange between multiple AI models and enable holistic health management and personalized engagement based on integrated insights from the AI models.

The plurality of subsystems also comprises a contact and data sharing subsystem configured to utilize near-field communication (NFC) to streamline user onboarding, enable the sharing of user profile data between devices, facilitate personalized marketing and community building, via a contact and data sharing subsystem.

The plurality of subsystems also comprises an AI voice interaction and personalized real-time feedback subsystem configured to recognize voice commands, employ AI to generate personalized recommendations, and deliver real-time feedback through natural language responses or auditory cues.

In accordance with one embodiment of the disclosure, a method for integrated fitness tracking and holistic health management using a computing system is disclosed. The comprises the steps of receiving user inputs regarding preferred activities and audio feedback, integrating with external music streaming services, and dynamically adjusting a user interface based on user preferences, using a customizable user experience subsystem of the computing system.

The method also includes tracking and recording fasting periods, physical activities, hydration, and sleep patterns, and providing rewards and incentives to users based on their health and fitness progress, using a holistic health management subsystem of the computing system.

The method also includes collecting and analyzing user-specific data, generating personalized workout plans and recommendations, and adapting plans based on changing user needs and progress, using an AI-driven personalization subsystem of the computing system.

The method also includes integrating with social media platforms to share user milestones, administering referral incentives and loyalty programs, and delivering targeted advertisements and promotions for fitness-related products and services, using an integrated marketing capabilities subsystem of the computing system.

The method also includes synchronizing real-time data from wearable devices and analyzing the data to provide detailed insights into fitness metrics, using a wearable device integration subsystem of the computing system.

The method also includes minimizing the environmental footprint of the system, encouraging users to adopt eco-friendly habits, and enhancing the energy efficiency of the system's operation, using a sustainable lifestyle promotion subsystem of the computing system.

The method also includes delivering targeted messages based on location using geofencing, facilitating community interaction and competition, and employing precision marketing tools to target specific user demographics, using a marketing and community engagement subsystem of the computing system.

The method also includes presenting a catalog of food, beverages, and fitness products, providing personalized product recommendations, facilitating secure shopping, checkout, and order management, and offering promotions, discounts, and subscription services, using an E-commerce subsystem of the computing system.

The method also includes managing physical beacons in retail locations, detecting user proximity to beacons, integrating with user data and AI to deliver personalized promotions and navigation, and providing retailers with data analytics for optimizing in-store strategies, using a beacon subsystem of the computing system.

The method also includes acquiring biometric data from wearable sensors, analyzing and interpreting biometric data, and generating personalized meal and hydration recommendations based on user data and biometric analysis, using a personalized meal suggestion subsystem of the computing system.

The method also includes collecting biometric and biomechanical data via sensors in wearable garments, transmitting data wirelessly to the computing system, and providing real-time feedback, personalized workout recommendations, and progress tracking, using a smart fabrics subsystem of the computing system.

The method also includes facilitating communication and data exchange between multiple AI models and enabling holistic health management and personalized engagement based on integrated insights from the AI models, using an AI model interoperability subsystem of the computing system.

The method also includes utilizing near-field communication (NFC) to streamline user onboarding, enable the sharing of user profile data between devices, and facilitate personalized marketing and community building, using a contact and data sharing subsystem of the computing system.

The method also includes recognizing voice commands, employing AI to generate personalized recommendations, and delivering real-time feedback through natural language responses or auditory cues, using an AI voice interaction and personalized real-time feedback subsystem of the computing system.

To further clarify the advantages and features of the present disclosure, a more particular description of the disclosure will follow by reference to specific embodiments thereof, which are illustrated in the appended figures. It is to be appreciated that these figures depict only typical embodiments of the disclosure and are therefore not to be considered limiting in scope. The disclosure will be described and explained with additional specificity and detail with the appended figures.

### BRIEF DESCRIPTION OF THE DRAWINGS

The disclosure will be described and explained with additional specificity and detail with the accompanying figures in which:

FIG. 1 is a block diagram illustrating various components of an exemplary computing system for integrated fitness tracking with holistic health management and AI-enhanced user engagement in accordance with an embodiment of the present disclosure;

FIG. 2 is a block diagram illustrating various components of an exemplary customizable user experience subsystem in accordance with an embodiment of the present disclosure;

FIG. 3 is a block diagram illustrating various components of an exemplary holistic health management subsystem in accordance with an embodiment of the present disclosure;

FIG. 4 is a block diagram illustrating various components of an exemplary AI-Driven personalization subsystem in accordance with an embodiment of the present disclosure;

FIG. 5 is a block diagram illustrating an exemplary integrated marketing capabilities subsystem in accordance with an embodiment of the present disclosure;

FIG. 6 is a block diagram illustrating an exemplary wearable device integration subsystem in accordance with an embodiment of the present disclosure;

FIG. 7 is a block diagram illustrating an exemplary sustainable lifestyle promotion subsystem in accordance with an embodiment of the present disclosure;

FIG. 8 is a block diagram illustrating an exemplary marketing and community engagement subsystem in accordance with an embodiment of the present disclosure;

FIG. 9 is a block diagram illustrating an exemplary e-commerce subsystem in accordance with an embodiment of the present disclosure;

FIG. 10 illustrates a block diagram illustrating an exemplary beacon subsystem in accordance with an embodiment of the present disclosure;

FIG. 11 illustrates a block diagram illustrating an exemplary personalized meal suggestion subsystem in accordance with an embodiment of the present disclosure;

FIG. 12 illustrates a block diagram illustrating an exemplary smart fabrics subsystem in accordance with an embodiment of the present disclosure;

FIG. 13 illustrates a block diagram illustrating an exemplary AI model interoperability subsystem in accordance with an embodiment of the present disclosure;

FIG. 14 illustrates a block diagram illustrating an exemplary contact and data sharing subsystem in accordance with an embodiment of the present disclosure;

FIG. 15 illustrates a block diagram illustrating an exemplary AI voice interaction and personalized real-time feedback subsystem in accordance with an embodiment of the present disclosure; and

FIG. 16A and FIG. 16B illustrates a block diagram illustrating an exemplary method for integrated fitness tracking with holistic health management and AI-enhanced user engagement in accordance with an embodiment of the present disclosure;

Further, those skilled in the art will appreciate that elements in the figures are illustrated for simplicity and may not have necessarily been drawn to scale. Furthermore, in terms of the construction of the device, one or more components of the device may have been represented in the figures by conventional symbols, and the figures may show only those specific details that are pertinent to understanding the embodiments of the present disclosure so as not to obscure the figures with details that will be readily apparent to those skilled in the art having the benefit of the description herein.

### DETAILED DESCRIPTION

In the following description of the embodiments of the invention, reference is made to the accompanying drawings that form a part hereof, and which are shown by way of illustration of specific embodiments in which the invention may be practiced. These embodiments are described in sufficient detail to enable those skilled in the art to practice the invention, and it is to be understood that other embodiments may be utilized and that changes may be made without departing from the scope of the present invention.

The specification may refer to “an”, “one” or “some” embodiment(s) in several locations. This does not necessarily imply that each such reference is to the same embodiment(s), or that the feature only applies to a single embodiment. Single feature of different embodiments may also be combined to provide other embodiments.

As used herein, artificial intelligence (AI) as used herein, refers to a machine learning system based on a deep neural network architecture. The AI system can adapt its parameters based on new inputs, enabling it to improve its performance over time

As used herein, the singular forms “a”, “an” and “the” are intended to include the plural forms as well unless expressly stated otherwise. It will be further understood that the terms “includes”, “comprises”, “including” and/or “comprising” when used in this specification, specify the presence of stated features, integers, steps, operations, elements and/or components, but do not preclude the presence or addition of one or more other features integers, steps, operations, elements, components, and/or groups thereof. As used herein, the term “and/or” includes any and all combinations and arrangements of one or more of the associated listed items.

Unless otherwise defined, all terms (including technical and scientific terms) used herein have the same meaning as commonly understood by one of ordinary skill in the art to which this disclosure pertains. It will be further understood that terms, such as those defined in commonly used dictionaries should be interpreted as having a meaning that is consistent with their meaning in the context of the relevant art and will not be interpreted in an idealized or overly formal sense unless expressly so defined herein.

The utility of the system described herein will be explained further in detail in the following sections of this document referring to the figures. Specific terms used herein do not restrict the scope of the present disclosure.

A computer system (standalone, client or server computer system) configured by an application may constitute a “subsystem” and “module” that is configured and operated to perform certain operations. In one embodiment, the “subsystem” or “module” may be implemented mechanically or electronically, so a subsystem or module may comprise dedicated circuitry or logic that is permanently configured (within a special-purpose processor) to perform certain operations. In another embodiment, a “subsystem” or “module” may also comprise programmable logic or circuitry (as encompassed within a general-purpose processor or other programmable processor) that is temporarily configured by software to perform certain operations.

Accordingly, the term “subsystem” or “module” should be understood to encompass a tangible entity, be that an entity that is physically constructed permanently configured (hardwired) or temporarily configured (programmed) to operate in a certain manner and/or to perform certain operations described herein.

Embodiments of the present invention disclose a system and method for integrated fitness tracking with holistic health management and AI-enhanced user engagement.

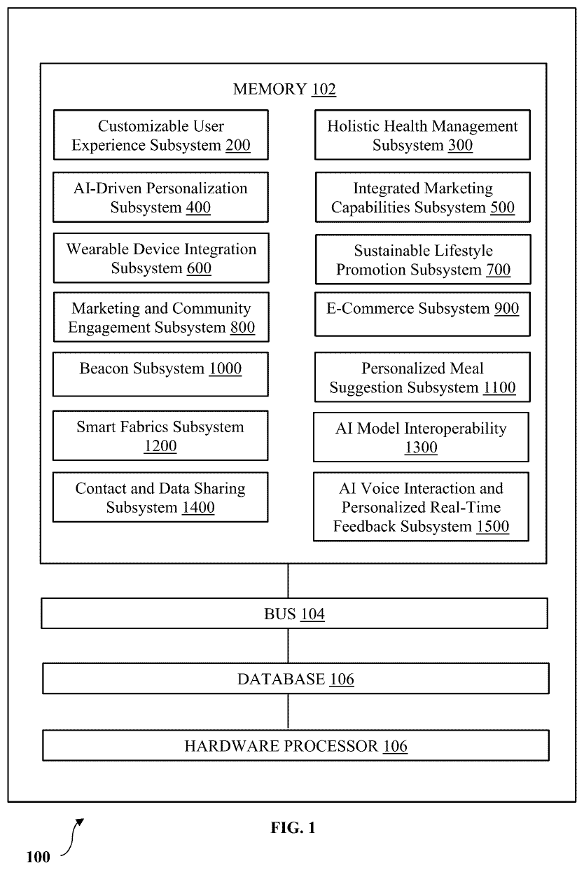

FIG. 1 is a block diagram illustrating various components of an exemplary computing system 100 for integrated fitness tracking with holistic health management and AI-enhanced user engagement in accordance with an embodiment of the present disclosure.

The computing system 100 includes a hardware processor 106. The computing system 100 also includes a memory 102 coupled to the hardware processor 106. The memory 102 comprises a set of program instructions in the form of a plurality of subsystems, modules and submodules configured to be executed by the hardware processor 106.

The hardware processor(s) 106, as used herein, means any type of computational circuit, such as, but not limited to, a microprocessor, a microcontroller, a complex instruction set computing microprocessor, a reduced instruction set computing microprocessor, a very long instruction word microprocessor, an explicitly parallel instruction computing microprocessor, a digital signal processor, or any other type of processing circuit, or a combination thereof.

The memory 102 includes a plurality of subsystems stored in the form of executable program which instructs the processor via bus to perform the method steps described below. The plurality of subsystems includes: a customizable user experience subsystem 200, a holistic health management subsystem 300, an AI-driven personalization subsystem 400, an integrated marketing capabilities subsystem 500, a wearable device integration subsystem 600, a sustainable lifestyle promotion subsystem 700, a marketing and community engagement subsystem 800, an E-commerce subsystem 900, a beacon subsystem 1000, a personalized meal suggestion subsystem 1100, a smart fabrics subsystem 1200, an AI model interoperability 1300, a contact and data sharing subsystem 1400 and an AI voice interaction and personalized real-time feedback subsystem 1500.

Computer memory elements may include any suitable memory device(s) for storing data and executable program, such as read only memory, random access memory, erasable programmable read only memory, electrically erasable programmable read only memory, hard drive, removable media drive for handling memory cards and the like. Embodiments of the present subject matter may be implemented in conjunction with program modules, including functions, procedures, data structures, and application programs, for performing tasks, or defining abstract data types or low-level hardware contexts. Executable program stored on any of the above-mentioned storage media may be executable by the hardware processor(s) 106.

The customizable user experience subsystem 200 is configured to receive user inputs regarding preferred activities and audio feedback, integrate with external music streaming services, and dynamically adjust a user interface based on user preferences.

The holistic health management subsystem 300 is configured to track and record fasting periods, physical activities, hydration, and sleep patterns, and provide rewards and incentives to users based on their health and fitness progress.

The AI-driven personalization subsystem 400 is configured to collect and analyze user-specific data, generate personalized workout plans and recommendations, and configured to adapt plans based on changing user needs and progress.

The integrated marketing capabilities subsystem 500 is configured to integrate with social media platforms to share user milestones, administer referral incentives and loyalty programs, and deliver targeted advertisements and promotions for fitness-related products and services.

The wearable device integration subsystem 600 is configured to synchronize real-time data from wearable devices and analyze the data to provide detailed insights into fitness metrics.

The sustainable lifestyle promotion subsystem 700 is configured to minimize the environmental footprint of the system, encourage users to adopt eco-friendly habits, and enhance the energy efficiency of the system's operation.

The marketing and community engagement subsystem 800 is configured to use geofencing to deliver targeted messages based on location, facilitate community interaction and competition, and employ precision marketing tools to target specific user demographics;

The E-commerce subsystem 900 is configured to present a catalog of food, beverages, and fitness products, provide personalized product recommendations, facilitate secure shopping, checkout, and order management, and offer promotions, discounts, and subscription services.

The beacon subsystem 1000 is configured to manage physical beacons in retail locations, detect user proximity to beacons, integrate with user data and AI to deliver personalized promotions and navigation, and provide retailers with data analytics for optimizing in-store strategies.

The personalized meal suggestion subsystem 1100 is configured to acquire biometric data from wearable sensors, analyze and interpret biometric data, and generate personalized meal and hydration recommendations based on user data and biometric analysis.

The smart fabrics subsystem 1200 is configured to incorporate sensors in wearable garments to collect biometric and biomechanical data, transmit data wirelessly to the computing system, and provide real-time feedback, personalized workout recommendations, and progress tracking.

The AI model interoperability subsystem 1300 is configured to facilitate communication and data exchange between multiple AI models and enable holistic health management and personalized engagement based on integrated insights from the AI models.

The contact and data sharing subsystem 1400 is configured to utilize near-field communication (NFC) to streamline user onboarding, enable the sharing of user profile data between devices, facilitate personalized marketing and community building, via a contact and data sharing subsystem.

The AI voice interaction and personalized real-time feedback subsystem 1500 is configured to recognize voice commands, employ AI to generate personalized recommendations, and deliver real-time feedback through natural language responses or auditory cues.

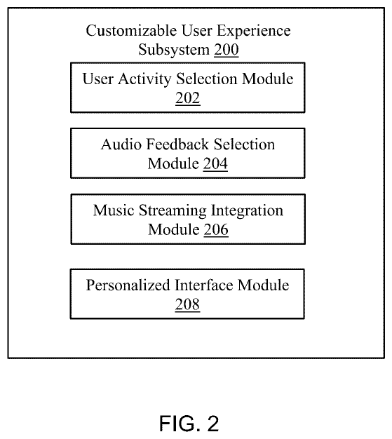

FIG. 2 is a block diagram illustrating various components of an exemplary customizable user experience subsystem 200 in accordance with an embodiment of the present disclosure. The customizable user experience subsystem 200 is configured to leverage advanced processing capabilities to collect and process user inputs, enabling a highly personalized and engaging workout environment. The customization process begins an user activity selection module 202 configured to receive user inputs regarding their preferred activities. Users can choose from a wide array of fitness activities, such as running, cycling, yoga, weight training, and more. This selection allows the application to tailor the exercise regimen to align with the user's interests and fitness goals, ensuring that each workout is enjoyable and motivating.

This allows users to customize their fitness journey by selecting preferred activities from a broad spectrum of options. Whether the user enjoys running, cycling, yoga, or strength training, the application provides a diverse array of activities to choose from, ensuring that every user can tailor their workout regimen to match their personal interests and fitness goals. This customization enhances user engagement and motivation by aligning the fitness program with individual preferences.

Next, an audio feedback selection module 204 is configured to receive user inputs related to their preferred audio feedback. Users can select from various audio options, comprising motivational cues, real-time live statistics, and progress updates. The audio feedback selection module 204 is configured to allow users to customize the auditory experience of their workouts, choosing the type of feedback that best keeps them motivated and informed. Motivational cues can provide encouragement and keep users inspired, while live statistics offer real-time data on their performance, helping them stay on track and achieve their fitness goals.

According to an embodiment of the present invention, the audio feedback selection feature offers users the ability to choose from various types of audio cues, including motivational messages, live statistics, and progress updates. This personalized audio feedback helps to keep users informed and motivated during their workouts, providing real-time encouragement and information that can enhance their overall exercise experience. Users can select the type of feedback that best suits their workout style and needs, creating a more engaging and supportive fitness environment.

Furthermore, the customizable user experience subsystem 200 provides a music streaming integration module 206 configured to incorporate user inputs for integrating external music streaming services. Users can select their preferred music streaming service, allowing them to access their favorite playlists and songs directly within the fitness application. This integration ensures that users can enjoy a seamless music experience during their workouts, enhancing their overall exercise experience. Music has been shown to significantly improve workout performance and enjoyment, making this feature a vital component of the personalized user experience.

According to an embodiment of the present invention, integrating external music streaming services into the customizable user experience subsystem 200 allows users to enjoy their favorite music during workouts. By connecting with popular music streaming platforms, users can create workout playlists, access curated fitness music, and enjoy seamless playback of their preferred tracks. This integration not only enhances the workout experience by adding a personal touch but also helps in maintaining the user's motivation and energy levels throughout their exercise sessions.

Further the customizable user experience subsystem 200 provides a personalized interface module 208 configured to dynamically adjusts the layout, design, and functionality of the UI to reflect the user's preferences. This personalized interface makes it easier for users to navigate the application, access their selected activities, audio feedback, and music, and stay engaged with their fitness journey. By creating a workout environment that is tailored to individual preferences, the system enhances user satisfaction, motivation, and adherence to fitness routines.

According to an embodiment of the present invention, the personalized interface adapts to the user's selections and preferences, providing a unique and customized workout environment. By dynamically adjusting the layout, themes, and functionalities of the application based on user input, the interface ensures a more intuitive and user-friendly experience. This customization makes it easier for users to navigate the application, track their progress, and stay engaged with their fitness goals.

Overall, the customizable user experience subsystem 200 of the computing system 100 offer a comprehensive approach to personalization. By allowing users to select their preferred activities, audio feedback, and music streaming services, and by adapting the user interface accordingly, the application creates a highly personalized and engaging workout environment. This customization not only improves user satisfaction but also promotes sustained engagement and success in achieving fitness goals.

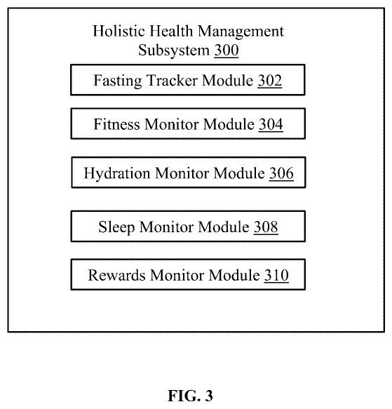

FIG. 3 is a block diagram illustrating various components of an exemplary holistic health management subsystem 300 in accordance with an embodiment of the present disclosure. The holistic health management subsystem 300 integrates various modules configured to monitor and enhance different aspects of a user's health and wellness, offering a rounded approach to fitness and well-being.

The first component of the holistic health management subsystem 300 is the fasting tracker module 302 configured to record and track users' fasting periods. The fasting tracker module 302 supports plurality of fasting protocols, allowing users to choose the one that best aligns with their lifestyle and health objectives. It provides tools to schedule fasting times, log fasting periods, and monitor adherence to fasting plans. By offering insights into fasting patterns and progress, the fasting tracker helps users optimize their fasting routines for improved metabolic health and overall wellness.

According to an embodiment of the present invention, the fasting tracker module 302 is configured to allow users to record and monitor their fasting periods. It provides tools to schedule fasting times, track fasting progress, and log fasting-related metrics. This module supports various fasting protocols, allowing users to choose the one that best fits their lifestyle and health goals. By providing insights into fasting patterns, the module helps users optimize their fasting routine for better health outcomes.

Next, the fitness monitor module 304 is configured to record and track physical activities. The fitness monitor module 304 configured to monitor a wide range of exercises, from running and cycling to strength training and yoga. It captures detailed metrics such as duration, intensity, calories burned, and performance trends. The fitness monitor enables users to set fitness goals, track their progress, and receive feedback on their physical activity levels. This continuous monitoring promotes consistent exercise habits and helps users achieve their fitness aspirations.

According to an embodiment of the present invention, the fitness monitor module 304 is configured to enable users to record and track their physical activities, including exercises, workouts, and daily movements. It provides detailed analytics on metrics such as duration, intensity, calories burned, and performance trends. The fitness monitor module 304 helps users stay on top of their physical activity levels, set fitness goals, and track their progress over time, promoting consistent and effective exercise habits.

The hydration monitor module 306 is another critical component, configured to track the user's water intake. Proper hydration is essential for maintaining optimal health and performance, and this module assists users in monitoring their daily water consumption. It allows users to log their water intake, set hydration goals, and receive timely reminders to drink water. By ensuring users stay properly hydrated, the hydration monitor supports overall health and enhances physical performance.

According to an embodiment of the present invention, the hydration monitor module 306 tracks the user's water intake, helping them maintain optimal hydration levels. Users can log their daily water consumption, set hydration goals, and receive reminders to drink water throughout the day. By monitoring hydration, this module ensures that users stay properly hydrated, which is crucial for overall health and effective physical performance.

Additionally, the sleep monitor module 308 is configured to record and analyze user sleep patterns. Good quality sleep is fundamental to health and recovery, and sleep monitor module 308 provides detailed insights into various aspects of sleep, including duration, sleep stages, and disturbances. It helps users understand their sleep quality and make necessary adjustments to improve their sleep hygiene. By promoting better rest and recovery, the sleep monitor contributes to enhanced physical and mental well-being.

According to an embodiment of the present invention the sleep monitor module 308 records and analyzes the user's sleep patterns, providing insights into sleep quality and duration. It tracks various sleep stages, detects sleep disturbances, and offers recommendations for improving sleep habits. By helping users understand their sleep patterns and make necessary adjustments, the sleep monitor module promotes better rest and recovery, contributing to overall health and well-being.

The final component is the rewards module 310, which is configured to motivate and engage users through a system of achievements and badges. This module tracks user progress against set challenges and personalized targets, awarding achievements and badges as users reach milestones. The gamification aspect of the rewards module adds an element of fun and motivation, encouraging users to stay committed to their health and fitness goals. By recognizing and celebrating user progress, this module enhances user engagement and fosters a sense of accomplishment.

According to an embodiment of the present invention, the rewards module 310 is configured to incentivize user engagement and progress through achievements and badges. Users can earn rewards based on their fitness milestones, challenge completions, and consistent adherence to their fitness routines. This gamification element adds a fun and motivating aspect to the fitness journey, encouraging users to stay committed and reach their health goals.

Overall, the holistic health management subsystem 300 integrates multiple modules that collectively address various aspects of health and wellness. The fasting tracker, fitness monitor, hydration monitor, sleep monitor, and rewards modules work together to provide users with comprehensive tools and insights to manage their health. This holistic approach not only supports users in achieving their fitness goals but also promotes overall well-being and a healthier lifestyle.

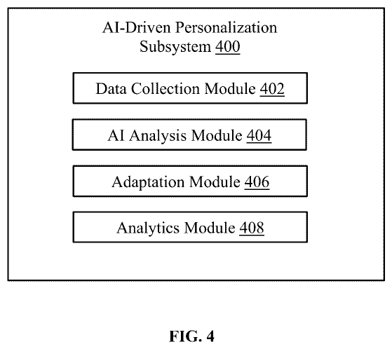

FIG. 4 is a block diagram illustrating various components of an exemplary AI-driven personalization subsystem 400 in accordance with an embodiment of the present disclosure. The AI-Driven personalization subsystem 400 is configured to leverages artificial intelligence to create a highly individualized fitness experience, ensuring that each user receives tailored workout plans and insights that align with their specific needs and goals.

Firstly, a data collection module 402 is configured to collect the user-specific data. This data encompasses a wide range of information, including the user's fitness history, activity preferences, and personal goals. Fitness history data includes past workouts, performance metrics, and progress trends, providing a comprehensive overview of the user's fitness journey. Activity preferences capture the types of exercises the user enjoys and engages in most frequently, whether it be cardio, strength training, yoga, or other activities. Personal goals detail what the user aims to achieve, such as weight loss, muscle gain, endurance improvement, or general health maintenance. By gathering this rich dataset, the system lays the foundation for precise and relevant personalization.

According to an embodiment of the present invention, the data collection feature gathers comprehensive user-specific data, including fitness history, activity preferences, personal goals, and biometric information. This data serves as the foundation for personalized fitness recommendations and insights. By collecting detailed user information, the application can tailor its services to meet individual needs and preferences, enhancing the overall user experience.

Further, an AI analysis module 404 is configured to process and analyse the collected user data. This AI analysis module 404 utilizes sophisticated algorithms and machine learning techniques to identify patterns and insights within the user data. The analysis phase involves understanding the user's current fitness level, identifying strengths and areas for improvement, and recognizing preferences and habits. Based on this analysis, the AI module generates personalized workout plans tailored to meet the user's unique needs and objectives. These plans include specific exercises, intensity levels, durations, and progression schemes that are best suited to help the user achieve their goals effectively and efficiently.

According to an embodiment of the present invention, the AI analysis module 404 processes the collected user data using advanced algorithms and machine learning techniques. It identifies patterns, trends, and correlations within the data to generate personalized workout plans and recommendations. The AI-driven insights help users optimize their fitness routines, address specific health concerns, and achieve their personal goals more effectively.

The AI-driven personalization subsystem 400 does not stop at initial personalization; it continuously adapts the workout plans based on the user's changing fitness needs. As the user progresses, logs workouts, and achieves milestones, an adaptation module 406 is configured to update the plans to reflect new data. This dynamic adaptation ensures that the workout plans remain relevant and challenging, preventing plateaus and promoting ongoing improvement. Whether the user experiences changes in fitness levels, shifts in goals, or evolves their preferences, the system responds by adjusting the workout plans accordingly, maintaining optimal alignment with the user's evolving fitness journey.

According to an embodiment of the present invention, this feature ensures that personalized workout plans and recommendations are continuously updated based on the user's changing fitness needs and progress. The adaptation module 406 dynamically adjusts the fitness plans to reflect new data, such as improvements in performance, changes in goals, or evolving preferences. This ongoing adaptation keeps the fitness program relevant and effective, supporting long-term success.

Finally, the AI-driven personalization subsystem 400 provides an analytics module 408 configured to provide a detailed insights into user behavior and preferences. This module offers comprehensive visualizations and reports that help users understand their fitness patterns, progress, and areas for improvement. By presenting data on workout frequency, performance trends, goal achievement, and more, the analytics module 408 empowers users to make informed decisions about their fitness routines. Additionally, these insights facilitate optimized service delivery by allowing the application to identify growth opportunities and areas where user experience can be enhanced. For example, understanding common user preferences can help in developing new features or refining existing ones to better meet user needs.

According to an embodiment of the present invention, the analytics module 408 provides detailed insights into user behavior, preferences, and performance metrics. It offers visualizations and reports that help users understand their fitness journey and make informed decisions. By providing actionable insights, the analytics module facilitates optimized service delivery and helps users identify areas for improvement and growth.

Overall, AI-driven personalization subsystem 400 provide a highly tailored and adaptive fitness experience. By collecting detailed user-specific data, utilizing AI to analyze this data and generate personalized workout plans, continuously adapting these plans based on changing fitness needs, and offering deep insights through an analytics module, the system ensures that users receive the most relevant, effective, and engaging fitness guidance. This personalized approach not only enhances user satisfaction and motivation but also drives better fitness outcomes and sustained engagement.

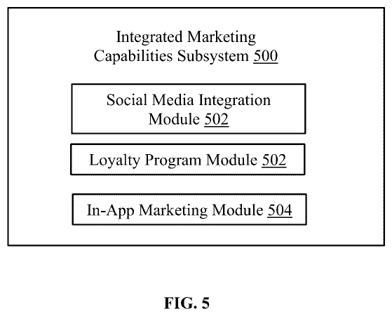

FIG. 5 is a block diagram illustrating an exemplary integrated marketing capabilities subsystem 500 in accordance with an embodiment of the present disclosure. The integrated marketing capabilities subsystem 500 enhances user engagement and expands the application's reach by incorporating modules designed to leverage social media, loyalty programs, and in-app marketing. These features work together to create a comprehensive marketing strategy that not only promotes user activity and retention but also generates additional revenue streams.

The first component of this system is the social media integration module 502. The social media integration module 502 is configured to seamlessly share user milestones and fitness achievements on various social media platforms. By enabling users to post their progress, such as completing a workout challenge, reaching a new personal best, or achieving a fitness goal, the module fosters a sense of accomplishment and community. This social sharing not only boosts user motivation and engagement but also serves as organic promotion for the fitness application. Friends and followers of the users who see these posts may be encouraged to join the application themselves, thereby increasing its user base.

According to an embodiment of the present invention, this feature allows users to share their fitness milestones, achievements, and progress on social media platforms. By integrating with popular social networks, the application enables users to celebrate their successes with friends and family, gain social support, and inspire others. Social media integration also helps build a community around the fitness journey, enhancing user engagement and motivation.

Next, the loyalty program module 504 plays a crucial role in encouraging user retention and growth. The loyalty program module 504 is configured to administer referral incentives, rewarding users for bringing new members to the application. It also manages participation in loyalty programs where users can earn points, badges, or other rewards for consistent use of the application, completing specific challenges, or achieving long-term fitness goals. These incentives create a gamified experience that motivates users to stay active and engaged with the application. The loyalty program not only enhances user satisfaction by recognizing and rewarding their efforts but also fosters a sense of loyalty and commitment to the fitness journey.

According to an embodiment of the present invention, the loyalty program module 504 configured to administer referral incentives and manages user participation in loyalty programs. Users can earn rewards for referring new members, completing challenges, and maintaining consistent activity. This module encourages user retention and growth by offering tangible benefits for continued engagement and participation in the fitness community.

The in-app marketing module 506 is configured to promotes fitness-related products, services, and supplements directly within the computing system 100. Leveraging user data and preferences, this module delivers targeted advertisements and offers that align with individual user needs and interests. For instance, a user who frequently engages in strength training might receive promotions for protein supplements, fitness equipment, or specialized workout gear. This targeted approach ensures that the advertisements are relevant and valuable to the users, increasing the likelihood of conversion. Additionally, the in-app marketing module 506 facilitate a significant revenue stream for the application by facilitating partnerships with fitness brands and retailers.

According to an embodiment of the present invention, the in-app marketing module 506 configured to promote fitness-related products, services, and supplements directly within the application. It leverages user data to deliver targeted advertisements and offers that align with individual preferences and needs. This feature provides users with relevant and useful recommendations, while also generating additional revenue streams for the application.

According to an embodiment of the present invention, the in-app marketing module 506 also seamlessly integrates the promotion of food and beverage products into the user experience. This module leverages user data, preferences, and activity patterns to deliver personalized and highly relevant marketing content, focusing specifically on promoting healthy dietary choices that align with users' fitness goals.

The in-app module 506 utilizes data analytics to understand individual user preferences, dietary habits, and fitness goals. Based on this data, it provides personalized recommendations for food and beverage products that support users' health and wellness objectives. For instance, a user focused on muscle building might receive suggestions for protein-rich snacks and supplements, while someone aiming for weight loss could be recommended low-calorie, nutritious meal options.

Further to enhance user trust and engagement, the in-app module 506 provides detailed nutritional information and health benefits of the promoted food and beverage products. Users can access information about calorie content, macronutrient breakdown (proteins, fats, carbohydrates), vitamins, and minerals, along with explanations of how these products can support their specific fitness goals. This educational approach helps users make informed dietary choices.

Further, the in-app module integrates seamlessly with the personalized workout plans generated by the AI-driven personalization system. It suggests food and beverage products that complement the user's fitness routine, ensuring that dietary recommendations are aligned with physical activity levels and nutritional needs. For example, after a high-intensity workout, the module might recommend a recovery drink rich in electrolytes and proteins to aid in muscle recovery and hydration.

To incentivize purchases, the in-app module offers exclusive promotional deals and discounts on healthy food and beverage products. Users can receive notifications about special offers, limited-time discounts, and bundle deals directly within the app. These promotions are tailored to user preferences and shopping behavior, increasing the likelihood of engagement and conversion.

The in-app marketing module 506 is also closely integrated with the loyalty program module 502, rewarding users for purchasing promoted food and beverage products. Users can earn points, badges, and other incentives for trying new products, participating in dietary challenges, and consistently making healthy dietary choices. This gamification element encourages users to stay engaged with the app and maintain healthy eating habits.

Further leveraging the geofencing, the in-app marketing module 506 can deliver location-based promotions for food and beverage products that are available. Users receive notifications about relevant deals and events when they are in the vicinity, making it convenient for them to access and purchase the promoted products.

Overall, the integrating marketing capabilities subsystem 500 create a synergistic approach to user engagement and revenue generation. The social media integration module enhances user motivation and broadens the application's reach through organic promotion. The loyalty program module drives user retention and activity by offering incentives and rewards. The in-app marketing module generates revenue and enhances user experience by delivering relevant promotions and advertisements. Together, these modules create a holistic marketing strategy that supports the application's growth and sustainability while enhancing user satisfaction and engagement.

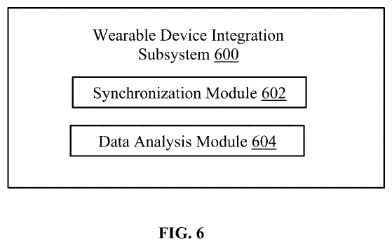

FIG. 6 is a block diagram illustrating an exemplary wearable device integration subsystem 600 in accordance with an embodiment of the present disclosure. The wearable device integration subsystem 600 is configured for integrating wearable devices with the computing system 100. This integration enhances the functionality and user experience of the computing system 100 by enabling seamless data synchronization and detailed analytics. By connecting fitness trackers and smartwatches to the computing system 100, users can access real-time data and insights, which contribute to a more comprehensive and personalized fitness experience.

The main feature of this wearable device integration subsystem 600 is the synchronization module 602, which is configured to receive and synchronize real-time data from connected wearable devices such as fitness trackers and smartwatches. The wearable device integration subsystem 600 ensures that data from various activities, such as walking, running, cycling, and other physical exercises, is accurately captured and transmitted to the fitness application. The synchronization process is continuous and automatic, providing users with up-to-date information without the need for manual data entry. This real-time data integration allows users to monitor their fitness metrics dynamically, enhancing their ability to track progress and adjust their activities accordingly.

According to an embodiment of the present invention, the synchronization module 602 is configured to enable real-time data exchange between the fitness application and connected wearable devices, such as fitness trackers and smartwatches. It ensures that activity data, biometric information, and other metrics are seamlessly integrated into the application. This real-time synchronization enhances the accuracy and comprehensiveness of fitness tracking.

Once the data is synchronized, it is processed by the data analysis module 604. The data analysis module 604 is configured employ techniques to analyze the incoming data and provide detailed insights into a range of fitness metrics. Key metrics include steps taken, calories burned, heart rate, distance covered, active minutes, and sleep patterns. The data analysis module 604 configured to provide comprehensive reports and visualizations that help users understand their physical activity levels and overall health status. For example, users can view their daily, weekly, and monthly step counts, track calorie expenditure trends, monitor heart rate variability, and assess the quality and duration of their sleep.

According to an embodiment of the present invention, data analysis module 604 is configured process the synchronized data from wearable devices to provide detailed insights into various fitness metrics, including steps taken, calories burned, heart rate, and more. By analyzing this data, the module helps users monitor their physical activity levels, track their progress, and make informed decisions about their fitness routines.

The insights generated by the data analysis module 604 are crucial for users aiming to achieve specific fitness goals. By providing a clear picture of their physical activities and health metrics, the module enables users to make informed decisions about their fitness routines. For instance, if a user notices a decline in their daily steps, they can set a goal to increase their activity level. Similarly, if the analysis indicates insufficient sleep, the user can take steps to improve their sleep hygiene. The detailed feedback and personalized recommendations derived from the analysis help users optimize their fitness strategies, enhance performance, and maintain overall well-being.

Furthermore, the integration of wearable devices adds an extra layer of convenience and motivation for users. The real-time feedback and detailed insights act as powerful motivators, encouraging users to stay active and engaged with their fitness journeys. Additionally, the ability to track progress over time and compare it against set goals fosters a sense of achievement and accountability, driving users to remain consistent and dedicated to their fitness routines.

Overall, the wearable device integration subsystem 600 enhances the user experience by providing seamless data synchronization and detailed analytics. The synchronization module ensures real-time data capture from connected devices, while the data analysis module processes this data to offer valuable insights into key fitness metrics. This integration empowers users with the information they need to monitor their progress, make informed decisions, and achieve their fitness goals effectively.

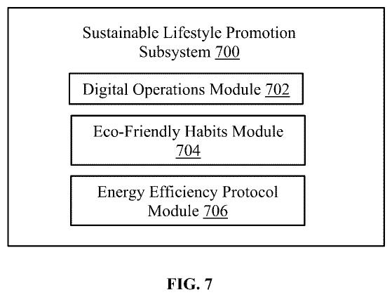

FIG. 7 is a block diagram illustrating an exemplary sustainable lifestyle promotion subsystem 700 in accordance with an embodiment of the present disclosure. This sustainable lifestyle promotion subsystem 700 integrates sustainability principles into the core functionalities of the application, encouraging users to adopt eco-friendly habits while minimizing the application's environmental impact. The comprehensive approach includes implementing modules focused on digital operations, eco-friendly practices, and energy efficiency, all designed to support a greener, more sustainable lifestyle.

The first component of the sustainable lifestyle promotion subsystem 700 is the digital operations module 702, which is configured to minimize the environmental footprint of the fitness application. The digital operations module 702 incorporates a range of strategies aimed at reducing digital waste and optimizing resource usage. For example, it may include features such as efficient data storage practices, reduced server energy consumption, and eco-friendly coding techniques that lower the computational load. By implementing these sustainable digital operations, the application ensures that its backend processes are as environmentally friendly as possible, contributing to a reduction in overall carbon emissions.

Next, the sustainable lifestyle promotion subsystem 700 provides an eco-friendly habits module 704 that is configured to actively encourage users to adopt sustainable practices in their daily lives. The eco-friendly habits module 704 promotes behaviors such as walking or cycling instead of driving, choosing plant-based meals, and practicing mindful eating. It offers tips, challenges, and incentives to motivate users to make these eco-conscious choices. For instance, the module might track the user's walking distance and compare it to the carbon emissions saved by not driving, providing tangible feedback on their positive environmental impact. Additionally, it can suggest sustainable dietary choices and highlight the environmental benefits of reduced meat consumption. By encouraging these practices, the eco-friendly habits module helps users lower their carbon footprint and embrace a more sustainable lifestyle.

The final component is the energy efficiency protocol module 706 which is configured to enhance the operational sustainability of the fitness application itself. This protocol involves a set of guidelines and practices aimed at reducing the energy consumption of the application's infrastructure. It may include optimizing server performance, using renewable energy sources for data centers, and implementing energy-saving features within the application. For example, the protocol might ensure that the application operates efficiently, reducing unnecessary background processes and conserving battery life on users' devices. By adopting these energy-efficient practices, the application not only lowers its own environmental impact but also sets an example for users, reinforcing the importance of sustainability.

Overall, the sustainable lifestyle promotion subsystem 700 integrates multiple modules that collectively address both the operational and user behavior aspects of sustainability. The digital operations module 702 focuses on minimizing the application's environmental footprint through efficient backend processes. The eco-friendly habits module 704 encourages users to adopt sustainable practices, providing tips and incentives for reducing carbon emissions. The energy efficiency protocol module 706 enhances the operational sustainability of the application by reducing energy consumption and optimizing resource usage. Together, these components create a holistic approach to promoting a greener, more sustainable lifestyle, benefiting both users and the environment.

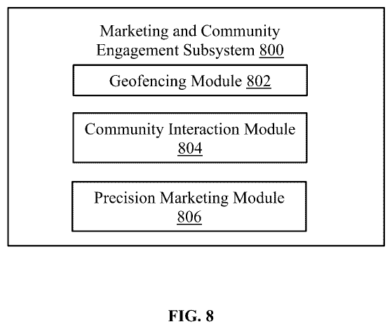

FIG. 8 is a block diagram illustrating an exemplary marketing and community engagement subsystem 800 in accordance with an embodiment of the present disclosure. The marketing and community engagement subsystem 800 is configured to enhance user interaction, drive engagement, and optimize marketing efforts through advanced features that leverage location data, social connectivity, and data analytics. The comprehensive approach includes modules focused on geofencing, community interaction, and precision marketing, all aimed at creating a more engaging and personalized user experience.

The first component of this marketing and community engagement subsystem 800 is the geofencing module 802, which is configured to deliver targeted advertisements and notifications based on the user's location. By utilizing geofencing technology, the module can identify when a user enters or exits a specific geographical area and trigger relevant marketing messages accordingly. For instance, users might receive notifications about nearby fitness events, special promotions at local gyms, or discounts at health food stores when they are in the vicinity. This location-based marketing ensures that advertisements are highly relevant and timely, increasing the likelihood of user engagement and conversion. The geofencing module 802 helps bridge the gap between digital and physical experiences, creating opportunities for users to connect with local fitness resources and communities.

According to an embodiment of the present invention, the geofencing module 802 is configured to deliver targeted advertisements and notifications based on the user's location. By leveraging geofencing technology, the application can provide location-specific offers, promotions, and reminders. This feature enhances the relevance and effectiveness of marketing efforts, increasing user engagement and satisfaction.

Next, the community interaction module 804 plays a crucial role in fostering a sense of connection and competition among users. The community interaction module 804 is configured to facilitate sharing, competition, and communication within an embedded social network. Users can share their fitness achievements, post updates, and engage with others through comments and likes. Additionally, the community interaction module 804 is configured support various forms of competition, such as leaderboards, fitness challenges, and group workouts, which encourage users to strive for their best and engage with the community. This social interaction not only motivates users to stay active and committed to their fitness goals but also creates a supportive environment where they can find encouragement and inspiration from their peers. By fostering a vibrant and interactive community, the module enhances the overall user experience and promotes long-term engagement.

According to an embodiment of the present invention, the community interaction module 804 facilitates sharing, competition, and communication among users within an embedded social network. It enables users to connect with others, join fitness challenges, and participate in community activities. The community interaction module fosters a sense of belonging and support, motivating users to stay active and engaged.

The final component is the precision marketing tool module 806, which utilizes data analytics to target specific user demographics and preferences. The precision marketing tool 806 is configured to analyze a wide range of user data, including age, gender, fitness goals, activity patterns, and past interactions with the application. Based on this analysis, it delivers personalized marketing messages that resonate with individual users. For example, a user who frequently participates in running might receive advertisements for running shoes, while someone focused on yoga might see promotions for yoga mats and classes. The precision marketing tool ensures that users receive relevant and valuable content, increasing the effectiveness of marketing campaigns and enhancing user satisfaction. By tailoring marketing efforts to match user preferences, the tool drives higher engagement and conversion rates.

According to an embodiment of the present invention, the precision marketing tool module 806 uses data analytics to target specific user demographics and preferences with tailored marketing campaigns. By analyzing user data, this tool delivers personalized advertisements and offers that resonate with individual users. This targeted approach maximizes the impact of marketing efforts, driving user engagement and conversion.

Overall, the marketing and community engagement subsystem 800 combine multiple modules to create a highly interactive and personalized user experience. The geofencing module 802 delivers location-based advertisements and notifications, connecting users with local fitness opportunities. The community interaction module 804 fosters sharing, competition, and communication, building a supportive and motivating social network. The precision marketing tool module 806 leverages data analytics to deliver targeted marketing messages that align with user demographics and preferences. Together, these components create a cohesive strategy that enhances user engagement, optimizes marketing efforts, and fosters a vibrant fitness community.

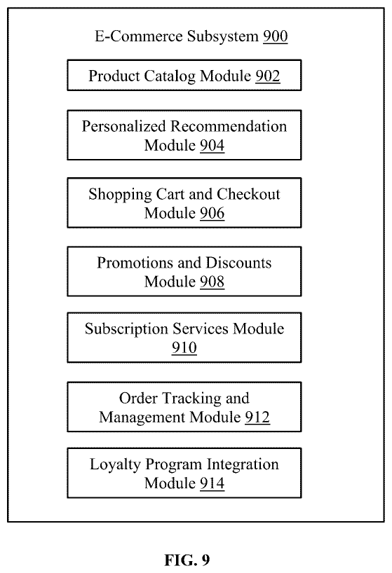

FIG. 9 is a block diagram illustrating an exemplary e-commerce subsystem 900 in accordance with an embodiment of the present disclosure. The e-commerce subsystem 902 is configured to provide users with a seamless and personalized shopping experience for food, beverages, and fitness-related products. This feature is structured into several modules, each focusing on a specific aspect of the e-commerce experience, ensuring comprehensive functionality and user satisfaction.

The e-commerce subsystem 902 provides a product catalog module 902 configured to showcase a wide variety of products relevant to health and fitness, including food items, beverages, supplements, fitness equipment, and apparel. Each product entry includes detailed descriptions, nutritional information, usage instructions, and high-quality images, ensuring that users have all the information they need to make informed purchasing decisions.

Next a personalized recommendation module 904 is disclosed, wherein the personalized recommendation module 904 is configured to employ artificial intelligence to analyze user data and provide customized product suggestions. By considering factors such as the user's fitness history, activity preferences, dietary goals, and previous purchase behavior, the module generates recommendations that are tailored to individual needs. For example, users aiming for muscle gain might see recommendations for high-protein foods and supplements, while those focusing on weight loss could receive suggestions for low-calorie snacks and meal replacements. This module ensures that users receive relevant and beneficial product suggestions that support their specific health and fitness objectives.

Further a shopping cart and checkout module 906 is configured to facilitate a smooth and secure purchasing process. Users can easily add products to their cart, view and modify their selections, and proceed to checkout. The checkout process includes several secure payment options, such as credit/debit cards and digital wallets, ensuring that users can complete their transactions safely. The module also provides a detailed order summary, including itemized costs, taxes, and any applied discounts. Users can review and confirm their orders before finalizing the purchase, ensuring transparency and accuracy.

Next a promotions and discounts module 908 is configured to offer users exclusive deals and savings opportunities. This module features personalized promotions based on user behavior and preferences, such as discounts on frequently purchased items or special offers on products that align with the user's fitness goals. Users receive notifications about flash sales, limited-time offers, and bundle discounts directly within the app. The module also supports the use of coupon codes, allowing users to apply additional savings at checkout. By providing targeted promotions, this module enhances user engagement and incentivizes purchases.

Also, subscription services module 910 is configured to enable users to subscribe to regular deliveries of their favorite products, such as meal kits, supplements, and workout gear. Users can set their preferred delivery intervals (e.g., weekly, bi-weekly, monthly) and manage their subscriptions through the app. This module ensures that users never run out of essential items and can benefit from subscription discounts. Users receive reminders before each delivery, allowing them to modify or pause their subscriptions as needed. The convenience of automatic deliveries helps maintain consistent use of health and fitness products.

Further an order tracking and management module 912 is configured provide users with real-time updates on their orders from placement to delivery. Users can track the status of their orders, receive notifications about shipping progress, and view estimated delivery times. The module includes a comprehensive order history, allowing users to review past purchases and reorder favorite items easily. This transparency and accessibility enhance user confidence and satisfaction by keeping them informed throughout the entire purchasing process.

Finally, a loyalty program integration module 912 is disclosed, wherein the loyalty program integration module is configured to reward users for their engagement and purchases within the app. Users earn loyalty points for every purchase, which can be accumulated and redeemed for discounts, free products, or exclusive rewards. The module tracks user progress within the loyalty program, providing clear information about points earned, available rewards, and user status (e.g., bronze, silver, gold tiers). By incentivizing repeat purchases and continuous engagement, the loyalty program fosters long-term user loyalty and satisfaction.

Overall, the modules collectively enhance the e-commerce functionality of the fitness application, providing a seamless, personalized, and rewarding shopping experience.

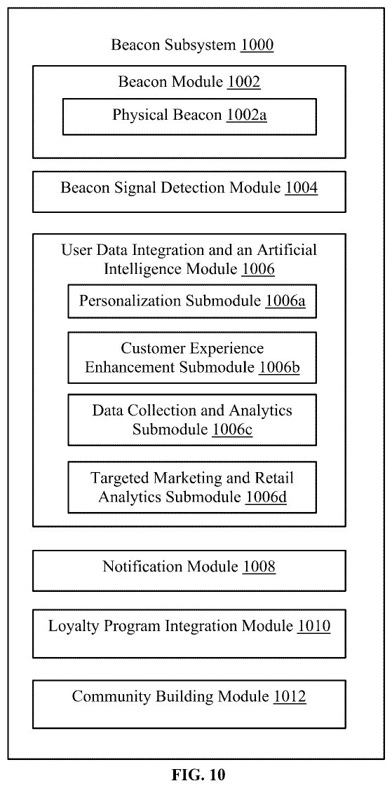

FIG. 10 illustrates a block diagram illustrating an exemplary beacon subsystem 1000 in accordance with an embodiment of the present disclosure. The beacon subsystem 1000 provides a beacon module 1002 configured to use one or more physical beacons 1002a in retail locations and the artificial intelligence techniques to create a highly personalized and engaging user experience, while also providing valuable insights to retailers for optimizing their marketing and sales strategies.

The beacon module 1002 module is configured to manage the physical beacons 1002a deployed in retail locations. These physical beacons 1002a emit signals that are detectable by user devices, such as smartphones. The beacon module 1002 ensures the physical beacons 1002a are functioning correctly, transmitting signals at the appropriate intervals, and are strategically placed to maximize their reach and impact.

The beacon subsystem 1000 also provides a beacon signal detection module 1004 integrated into the beacon subsystem 1000, this module detects signals from the beacons 1002a when a user is in proximity to a retail location. It communicates with the user's device to determine their location and proximity to specific beacons 1002a, enabling the system to trigger relevant actions and notifications based on their whereabouts.

The beacon subsystem 1000 also comprises a user data integration module and artificial intelligence module 1006 configured to access user data from the beacon subsystem 1000, including fitness goals, activity levels, purchase history, and preferences. This data is crucial for personalizing the user experience and tailoring recommendations based on individual needs and interests.

Further the user data integration module and artificial intelligence module 1006 comprises a personalization submodule 1006a, a customer experience enhancement submodule 1006b, a data collection and analytics submodule 1006c, and a targeted marketing module retail analytics submodule 1006d.

The personalization submodule 1006a is configured to analyzes user data and beacon signals to generate personalized promotions, product recommendations, and in-store navigation guidance. This ensures that users receive relevant and timely information that aligns with their fitness goals and preferences.

The customer experience enhancement submodule 1006b is configured to facilitate seamless mobile payments, check-ins, and rewards redemption at retail locations. This enhances the overall shopping experience and encourages users to interact with partner stores.

The data collection and analytics submodule 1006c is configured to collect data on user interactions with beacons and in-store behaviors. This data provides valuable insights to retailers, helping them optimize store layout, inventory management, and marketing strategies.

The targeted marketing module retail analytics submodule 1006d is configured to utilizes user data and beacon signals to deliver targeted marketing messages and offers based on user location and previous interactions with the system 1000. This ensures that users receive promotions that are relevant to their interests and current location. Further, it provides retailers with analytics on product interactions, sales performance, and the effectiveness of in-store promotions. This data enables data-driven marketing strategies, helping retailers maximize their sales and customer engagement.

The beacon subsystem 1000 also comprises a notification module 1020 configured to delivers personalized notifications, promotions, and rewards to users based on their proximity to retail locations and their interaction with beacons. This creates a sense of immediacy and relevance, encouraging users to take advantage of in-store offers and events.

Further a loyalty program integration module 1022 is configured to integrates with the computing system's 100 loyalty program feature, automatically awarding points to users for visiting partner stores or purchasing recommended products. This reinforces positive behaviors and encourages continued engagement with the app and partner retailers.

Also, a community building module 1024 is configured to facilitate social connections among users by notifying them of nearby fitness events or group workouts. This helps build a sense of community and encourages users to engage with others who share similar fitness interests.

Overall, the beacon subsystem 1000 enhances user engagement, provides valuable data to retailers and fosters a sense of community among users. By leveraging beacons, user data, and artificial intelligence, this subsystem creates a personalized and rewarding fitness experience that benefits both users and retailers.

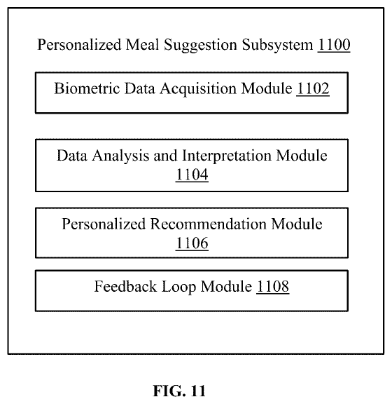

FIG. 11 illustrates a block diagram illustrating an exemplary personalized meal suggestion subsystem 1100 in accordance with an embodiment of the present disclosure. The personalized meal suggestion subsystem 1100 is configured to use biometric data and user profile information to provide customized meal and hydration recommendations, promoting optimal nutrition and performance for users.

The personalized meal suggestion subsystem 1100 provides a biometric data acquisition module 1102 configured to interface with one or more wearable biometric sensors, such as a fitness tracker or smartwatch, to collect sweat composition data from the user during physical activity or throughout the day. The sensor measures biomarkers indicative of hydration status and electrolyte balance, including sodium, potassium, and glucose levels. This real-time data provides valuable insights into the user's physiological state and nutritional needs.

The personalized meal suggestion subsystem 1100 further provides a data analysis and interpretation module 1104 is configured to use AI techniques to analyze and interpret the biometric data from the biometric data acquisition module 1102. It identifies patterns, trends, and correlations to assess the user's nutritional state, such as dehydration, electrolyte imbalances, or potential deficiencies. This analysis forms the basis for generating personalized meal suggestions.

The personalized meal suggestion subsystem 1100 further provides a personalized recommendation module 1106, wherein this module is configured to integrate user profile data, including dietary preferences, allergies, fitness goals, and activity levels, with the analyzed biometric data. It then generates personalized meal and hydration suggestions tailored to address the user's identified nutritional needs. These recommendations may include specific food items, beverages, or electrolyte supplements designed to replenish lost nutrients, maintain optimal hydration, and support the user's fitness goals. The engine also adapts meal suggestions in real-time based on the user's current activity level, ensuring that recommendations are aligned with their energy expenditure and physiological demands.

The personalized meal suggestion subsystem 1100 further provides a feedback loop module 1108 configured to allow users to provide feedback on the meal and hydration suggestions they receive. This feedback is incorporated into the AI techniques used by the recommendation engine, enabling it to refine and improve future recommendations based on user preferences and experiences. This continuous feedback loop ensures that the suggestions become increasingly accurate and tailored to the individual over time.

Overall, the personalized meal suggestion subsystem 1100 provides a comprehensive and dynamic approach to nutritional guidance. By integrating biometric data, user preferences, and real-time activity levels, this subsystem delivers customized meal and hydration recommendations that optimize user health, performance, and overall well-being.

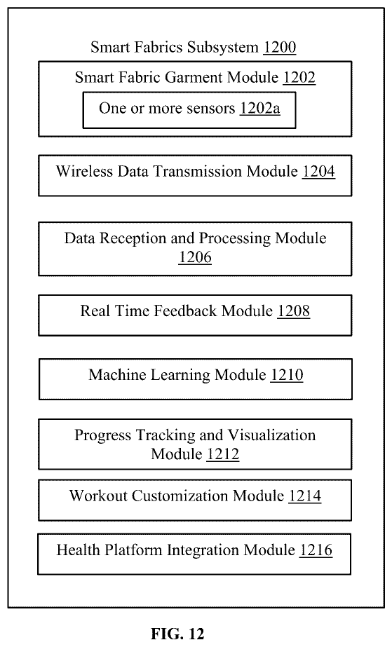

FIG. 12 illustrates a block diagram illustrating an exemplary smart fabrics subsystem 1200 in accordance with an embodiment of the present disclosure. The smart fabrics subsystem 1200 leverages advanced wearable technology and machine learning to provide users with real-time feedback, personalized workout recommendations, and comprehensive progress tracking.

It provides a smart fabric garment module 1202 is configured to encompass the wearable garment itself, incorporating a network of sensors 1202a embedded into the fabric. These sensors 1202a are designed to detect and measure various physiological and biomechanical metrics, including heart rate, muscle strain, temperature, and movement patterns. Specifically, the sensors can detect muscle activation levels, allowing for targeted feedback on muscle engagement during exercises.

The smart fabrics subsystem 1200 further provides a wireless data transmission module 1204 configured to integrated into the smart fabric garment, this module transmits sensor data wirelessly to a paired user device, such as a smartphone, running the fitness application. This ensures a seamless and real-time flow of data from the garment to the application for analysis and interpretation.

The smart fabrics subsystem 1200 further provides a data reception and processing module 1206 configured to receive the real-time sensor data from the smart fabric garment and employs advanced algorithms to analyze and interpret the data. It assesses exercise form, posture, and overall performance, providing insights into the user's movement patterns and potential areas for improvement.

The subsystem 1200 further comprises a real-time feedback module 1208 configured to provide immediate feedback to the user through visual, auditory, or haptic cues within the fitness application. It alerts users to incorrect form, posture deviations, or potential injury risks, offering suggestions for movement corrections and technique improvement. This real-time feedback enhances exercise effectiveness and safety.

The subsystem 1200 further comprises a machine learning module 1210 configured to use machine learning techniques to continuously analyze user data and movement patterns. Over time, it learns individual characteristics and provides increasingly personalized guidance, adapting feedback and recommendations to the user's specific needs and progress.

The subsystem 1200 further comprises a progress tracking and visualization module 1212 configured to track and records user performance data over time, allowing users to monitor their progress and identify areas for improvement. It presents this data through intuitive visualizations within the fitness application, such as charts and graphs, making it easy for users to understand their performance trends.

The subsystem 1200 further comprises a workout customization module 1214, configured to generate personalized workout plans based on the analysis of smart fabric data and user goals. These plans are tailored to target specific muscle groups, address areas of weakness, and optimize training based on the user's unique movement patterns and capabilities.

The subsystem 1200 further comprises a health platform integration module 1216 is configured to allow seamless sharing of smart fabric data with other health and fitness platforms. This integration enables users to aggregate data from multiple sources, creating a comprehensive overview of their health and fitness status.

Overall, the smart fabrics subsystem 1200 represents a significant advancement in wearable fitness technology. By combining smart fabric sensors, real-time data transmission, advanced analytics, and machine learning, this subsystem provides a highly personalized and effective fitness experience. Users receive immediate feedback on their form and technique, personalized workout recommendations, and detailed progress tracking, all contributing to improved performance, safety, and motivation.

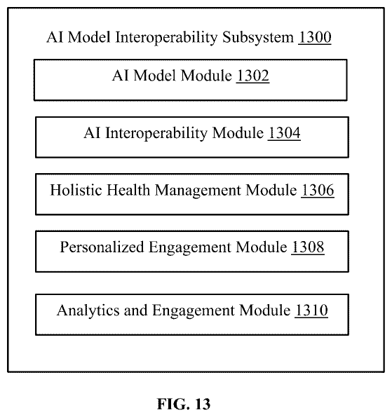

FIG. 13 illustrates a block diagram illustrating an exemplary AI model interoperability subsystem 1300 in accordance with an embodiment of the present disclosure. The AI model interoperability subsystem 1300 is configured to facilitate seamless communication and collaboration between multiple AI models, harnessing their collective intelligence to provide users with a more comprehensive and personalized fitness experience.

The subsystem 1300 provides an AI model module 1302 configured to provide plurality of AI models, each specialized in analyzing different types of user data. These models process and interpret fitness activity tracking data, biometric data (heart rate, sleep patterns), nutrition and hydration data, user preferences and demographics, purchase history, and product interactions. By analyzing diverse data sources, the AI models gain a holistic understanding of the user's health, fitness, and lifestyle.

The subsystem 1300 also provides an AI interoperability module 1304 configured to provide act as the central hub for communication and data exchange between the various AI models. It enables each model to access and utilize relevant data from other models, fostering collaboration and enhancing their individual analysis and decision-making capabilities. For instance, the fitness tracking AI model could share data with the nutrition AI model to generate personalized meal plans that complement the user's exercise routines. This interoperability amplifies the system's ability to provide comprehensive and tailored recommendations.

The subsystem 1300 also provides a holistic health management module 1306 configured to receive integrated insights from the AI interoperability module 1304, leveraging the combined analysis of multiple data sources. Based on this comprehensive understanding of the user, it generates personalized fitness plans, nutrition recommendations, and hydration strategies. This holistic approach ensures that recommendations are not only tailored to individual needs but also consider the interconnectedness of various aspects of health and wellness.

The subsystem 1300 further provides a personalized engagement module 1308 configured to access the interoperable AI insights to create a highly personalized user experience. It tailors marketing messages and promotions, recommends relevant products (e.g., Liquid Hydration products), customizes community interactions and social networking features, and adapts content and recommendations based on evolving user needs and preferences. By delivering a user-centric experience, this module enhances engagement and fosters long-term user satisfaction.

The subsystem 1300 further provides an analytics and improvement module 1310 configured to continuously collect and analyze data on user interactions with the fitness application and its various features. It provides valuable insights into user behavior, engagement patterns, and the effectiveness of personalized recommendations. This data-driven approach enables continuous improvement of the AI models and the overall user experience, ensuring that the system remains adaptive and responsive to user needs.

Overall, the AI model interoperability subsystem 1300 revolutionizes the fitness app landscape by enabling seamless collaboration between multiple AI models. This synergistic approach allows the system to provide users with comprehensive, personalized, and data-driven recommendations that optimize their health and fitness journeys. The continuous analysis of user data and feedback ensures that the system remains adaptive and responsive, delivering a truly tailored experience that evolves with the user's needs.

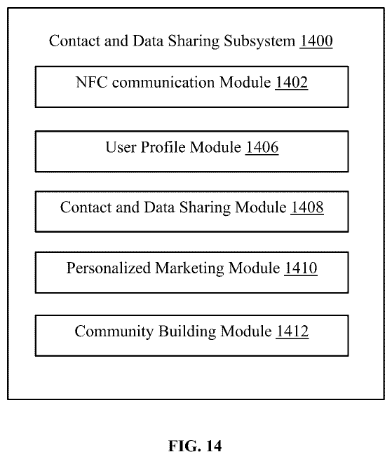

FIG. 14 illustrates a block diagram illustrating an exemplary contact and data sharing subsystem 1400 in accordance with an embodiment of the present disclosure. The contact and data sharing subsystem 1400 is configured to utilize near-field communication (NFC) technology to streamline user onboarding, enable seamless data sharing, enhance personalized marketing, and foster a vibrant fitness community.

The subsystem 1400 comprises a NFC communication module 1402 configured to integrate the subsystem 1400 on user devices and enable the establishment of NFC connections with other compatible devices. It facilitates the rapid exchange of data between devices in close proximity, enhancing the overall user experience.

The NFC communication module 1402 also configured to supports streamlined onboarding for new users. By establishing an NFC connection with an existing user's device, new users can quickly import relevant system data or personalized settings, eliminating the need for manual setup. Additionally, this module enables users to easily share loyalty program information or discount codes at retail locations equipped with NFC readers, streamlining the redemption process.

The subsystem 1400 further comprises a user profile module 1404 configured to store essential user data, including contact information (name, email, phone number), fitness goals, activity levels, preferences, and purchase history. This data is crucial for personalizing the user experience and facilitating data sharing between users.

The subsystem 1400 further comprises a contact and data sharing module 1406 configured to be activated upon detection of an NFC connection with another device running the fitness application. It facilitates the bidirectional exchange of selected user profile data between devices, including contact information, profile information (fitness level, goals), fitness achievements or milestones, personalized system settings, loyalty program information, and reward codes. This seamless data sharing enhances user interaction and community building.

The subsystem 1400 further comprises a personalized marketing module 1408 configured to access shared contact information and user data to deliver targeted marketing messages and promotions. For instance, users may receive offers for Liquid Hydration products aligned with their fitness goals or invitations to fitness events or group workouts. This personalized marketing approach ensures that users receive relevant and valuable content.

The subsystem 1400 further comprises a community building module 1410 configured to facilitate social interaction between users who have shared contact information through NFC. It enables the creation of in-app connections or friend lists, allowing users to share fitness achievements, progress updates, and participate in group challenges or competitions. This feature promotes a sense of community and camaraderie among users, enhancing motivation and engagement.

Overall, the contact and data sharing subsystem 1400 leverages NFC technology to streamline user onboarding, enable seamless data exchange between users, deliver personalized marketing, and foster community building. By facilitating effortless interaction and data sharing, this subsystem enhances the overall user experience and encourages engagement with the fitness application and its community features.

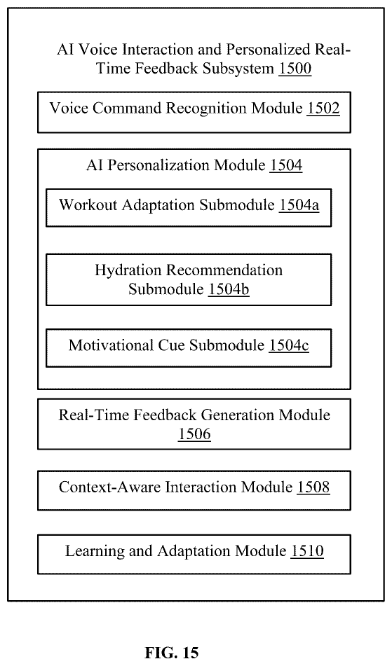

FIG. 15 illustrates a block diagram illustrating an exemplary AI voice interaction and personalized real-time feedback subsystem 1500 in accordance with an embodiment of the present disclosure. The AI voice interaction and personalized real-time feedback subsystem 1500 is configured to utilize voice recognition, artificial intelligence, and real-time data analysis to provide users with personalized guidance, motivation, and adjustments during their workouts.

This subsystem 1500 comprises a voice command recognition module 1502 configured to integrate with the user's device microphone to capture voice commands. It employs natural language processing (NLP) techniques to interpret user intent and extract relevant information from spoken commands. This module goes beyond recognizing predefined keywords, allowing for more natural and conversational interactions with the user.

This subsystem 1500 further comprises an AI personalization module 1504 configured to access and analyzes user data, including fitness goals, activity levels, historical performance, and real-time biometric data like heart rate and pace. Utilizing machine learning, it adapts to individual user preferences and needs, generating personalized workout plans, exercise modifications, hydration recommendations, and motivational cues based on real-time performance data and user goals.

The AI personalization module 1504 further comprises a workout adaptation submodule 1504a configured to dynamically adjusts workout intensity, duration, or exercise selection based on the user's real-time performance and feedback. It ensures that the workout remains challenging and effective while preventing overexertion or injury.

The AI personalization module 1504 further comprises a hydration recommendation submodule 1504b configured to analyze the user's biometric data, such as sweat rate and electrolyte levels, to provide personalized hydration recommendations. It suggests appropriate fluid intake and timing to maintain optimal hydration during workouts.

The AI personalization module 1504 further comprises a motivational cue submodule 1506b configured to deliver motivational messages and cues based on the user's performance, goals, and preferences. These cues aim to boost motivation, encourage persistence, and enhance the overall workout experience.

The AI voice interaction and personalized real-time feedback subsystem 1500 further comprises a real-time feedback generation module 1512 configured to receive output from the AI-driven personalization engine and synthesizes natural language responses or auditory cues based on personalized recommendations. It delivers real-time feedback to the user through the device's speaker, offering guidance, encouragement, and adjustments to the workout routine as needed.

The subsystem 1500 also comprises a context-aware interaction module 1514 configured to maintains a contextual understanding of the user's ongoing workout session. It ensures that responses and suggestions are relevant to the current activity and user state, providing timely and appropriate feedback.

The subsystem 1500 further comprises a learning and adaptation module 1516 configured to continuously learns from user interactions and feedback, refining the AI model's ability to understand user commands and preferences. Over time, it improves the accuracy and personalization of real-time feedback, creating a more tailored and effective workout experience.

Overall, the AI voice interaction and personalized real-time feedback subsystem 1500 represents a significant leap in fitness technology. By combining voice recognition, AI-driven personalization, and real-time data analysis, this subsystem provides users with a virtual coach that adapts to their individual needs and guides them towards their fitness goals. This interactive and personalized approach enhances motivation, optimizes performance, and fosters a more engaging and rewarding workout experience.

According to an embodiment of the present invention, a move-to-earn feature is provided that rewards users for engaging in physical activities such as walking, running, biking, or swimming. The system integrates with users' fitness trackers and wearable devices to monitor their activities and convert them into digital rewards. Users can sync their fitness trackers or wearable devices with the application to track physical activities in real-time. The system supports a wide range of devices, including smartwatches, fitness bands, and smartphone apps. As users engage in physical activities, the application tracks their progress and converts it into rewards, such as cryptocurrency tokens or non-fungible tokens (NFTs). These rewards can be accumulated, traded, or redeemed for various goods and services within the system. Additionally, users can join fitness challenges and competitions sponsored by the move-to-earn platform, enhancing engagement and motivation. Winners of these challenges receive additional rewards, promoting a competitive and social fitness environment.

Another embodiment expands the system's capabilities by integrating with popular move-to-earn apps like Sweatcoin, ActFit, FitCoin, LifeCoin, Wellcoin, and Step App (FITFI). This integration allows users to earn rewards across multiple platforms seamlessly. The system enables users to link their accounts from various move-to-earn apps, synchronizing their activities and rewards in one unified interface. Users can track their overall progress and rewards across different platforms without switching between apps. A centralized dashboard displays the user's cumulative rewards from all integrated apps, providing a comprehensive overview of their earnings. Users can manage their rewards, including saving, trading, or redeeming them for goods and services. The system can form partnerships with move-to-earn app providers to offer exclusive rewards and challenges to users. Collaboration with high-profile athletes or celebrities, such as the partnership with Olympic athlete Usain Bolt for the Step App, enhances user engagement and platform visibility.

Another embodiment of the present invention focuses on leveraging financial incentives and virtual fitness communities to boost user motivation and accountability, encouraging healthier habits and sustained physical activity. The system provides financial rewards in the form of cryptocurrency tokens or NFTs for every tracked activity, making fitness more appealing and rewarding. Users can set personal fitness goals and receive incremental rewards as they achieve milestones, fostering continuous motivation. The application includes social features that allow users to connect with friends, join virtual fitness communities, and participate in group challenges. Community support and social interactions help users stay accountable, motivated, and engaged in their fitness journey. The system offers personalized insights and recommendations based on users' activity data, helping them improve their fitness routines and overall well-being. Integration with health data, such as sleep patterns and nutrition, provides a holistic view of the user's health, encouraging balanced and sustainable lifestyle changes.

The benefits of incorporating move-to-earn features and integrating with popular apps are significant. Financial incentives make fitness activities more appealing, encouraging users to stay active and earn rewards for their efforts. Virtual fitness communities and social interactions help users stay accountable to their fitness goals while earning extra rewards. Integrating fitness and cryptocurrency promotes overall well-being by bridging the gap between physical activity and financial rewards. By incorporating move-to-earn features and integrating with popular apps, the system offers a comprehensive and engaging approach to fitness tracking and holistic health management. Users are motivated to stay active through financial rewards and community support, promoting healthier habits and overall well-being.

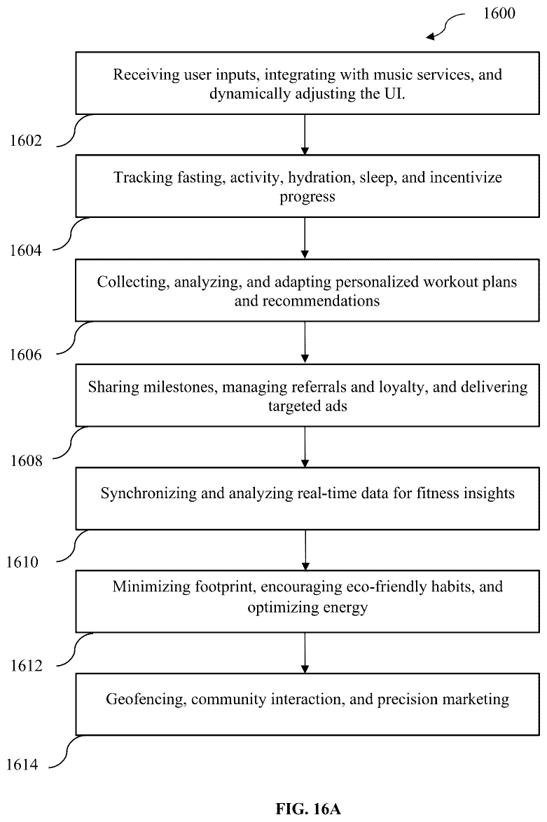

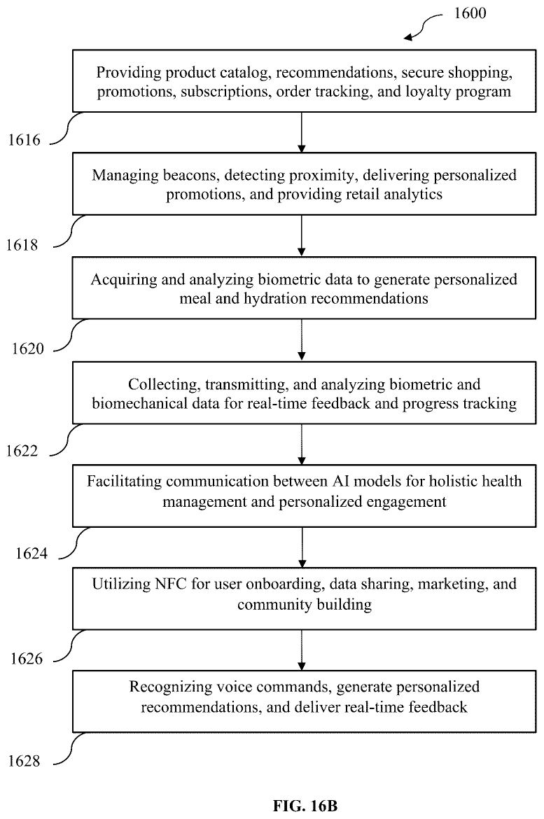

FIG. 16A and FIG. 16B illustrates a block diagram illustrating an exemplary method 1600 for integrated fitness tracking with holistic health management and AI-enhanced user engagement in accordance with an embodiment of the present disclosure. A method for integrated fitness tracking and holistic health management using a computing system is disclosed. The method comprises at step 1602, receiving user inputs regarding preferred activities and audio feedback, integrating with external music streaming services, and dynamically adjusting a user interface based on user preferences, using a customizable user experience subsystem of the computing system.

According to an embodiment of the present disclosure, the step of dynamically adjusting the user interface further comprises modifying the layout and color scheme of the user interface based on user-selected activity type, displaying preferred audio feedback types selected by the user during the activity and integrating the user interface with a selected external music streaming service for playback during the activity.

Next at step 1604, the method 1600 involves tracking and recording fasting periods, physical activities, hydration, and sleep patterns, and providing rewards and incentives to users based on their health and fitness progress, using a holistic health management subsystem of the computing system.

According to an embodiment of the present disclosure, the step of tracking and recording fasting periods further comprises receiving user input regarding the start and end times of a fasting period, calculating and displaying the duration of the fasting period and tracking the frequency and pattern of fasting periods over time.

Next at step 1606, the method 1600 involves collecting and analyzing user-specific data, generating personalized workout plans and recommendations, and adapting plans based on changing user needs and progress, using an AI-driven personalization subsystem of the computing system.

Next at step 1608, the method 1600 involves integrating with social media platforms to share user milestones, administering referral incentives and loyalty programs, and delivering targeted advertisements and promotions for fitness-related products and services, using an integrated marketing capabilities subsystem of the computing system.

Next at step 1610, the method 1600 involves synchronizing real-time data from wearable devices and analyzing the data to provide detailed insights into fitness metrics, using a wearable device integration subsystem of the computing system.

Next at step 1612, the method 1600 involves minimizing the environmental footprint of the system, encouraging users to adopt eco-friendly habits, and enhancing the energy efficiency of the system's operation, using a sustainable lifestyle promotion subsystem of the computing system.

Next at step 1614, the method 1600 involves delivering targeted messages based on location using geofencing, facilitating community interaction and competition, and employing precision marketing tools to target specific user demographics, using a marketing and community engagement subsystem of the computing system.

Next at step 1616, the method 1600 involves presenting a catalog of food, beverages, and fitness products, providing personalized product recommendations, facilitating secure shopping, checkout, and order management, and offering promotions, discounts, and subscription services, using an E-commerce subsystem of the computing system.

According to an embodiment of the present invention, the step of monitoring and recording physical activities further comprises receiving data from wearable devices or user input regarding exercise type, duration, intensity, and distance, calculating calories burned and other relevant metrics based on the received activity data and providing feedback to the user on their activity levels and progress toward fitness goals.

According to an embodiment of the present invention, the step of monitoring and recording user hydration levels further comprises receiving user input regarding water intake throughout the day, calculating and displaying daily, weekly, or monthly hydration levels based on the user input and sending reminders to the user to maintain adequate hydration.

According to an embodiment of the present invention, the step of monitoring and analyzing sleep patterns further comprises receiving data from wearable devices or user input regarding sleep duration and quality, analyzing sleep data to identify sleep stages and disturbances and providing feedback and recommendations to the user on improving sleep hygiene.

According to an embodiment of the present invention, the step of providing rewards and incentives further comprises defining fitness goals for the user, such as step count, calories burned, or exercise frequency, tracking the user's progress towards the defined goals and awarding virtual badges, achievements, or points upon reaching specific milestones.

According to an embodiment of the present invention, the step of integrating with social media platforms further comprises enabling users to connect their social media accounts to the computing system, allowing users to share workout summaries, achievements, and progress updates directly to their social media profiles and facilitating social interaction and engagement through comments, likes, and shares on social media posts.

According to an embodiment of the present invention, the step of administering referral incentives and loyalty programs further comprises generating unique referral codes for each user, tracking successful referrals made by users, rewarding users with incentives such as discounts, premium features, or in-app currency for each successful referral and assigning points or rewards to users based on their participation in the loyalty program.

According to an embodiment of the present invention, the step of delivering targeted advertisements and promotions further comprises analyzing user data to identify preferences, demographics, and interests, selecting and displaying relevant advertisements or promotions based on the analyzed user data and tracking user engagement with the advertisements and promotions.

According to an embodiment of the present invention, the step of presenting a catalog of food, beverages, and fitness products further comprises displaying product descriptions, nutritional information, images, and prices, allowing users to browse and search for products by category, brand, or keyword and updating the product catalog dynamically based on inventory and promotional offers.

Next at step 1618, the method 1600 involves managing physical beacons in retail locations, detecting user proximity to beacons, integrating with user data and AI to deliver personalized promotions and navigation, and providing retailers with data analytics for optimizing in-store strategies, using a beacon subsystem of the computing system.

According to an embodiment of the present invention, the step of managing physical beacons in retail locations further comprises configuring and maintaining the operational parameters of beacons, such as signal strength and frequency, monitoring the battery status and connectivity of beacons and updating beacon firmware and software as needed.

Next at step 1620, the method 1600 involves acquiring biometric data from wearable sensors, analyzing and interpreting biometric data, and generating personalized meal and hydration recommendations based on user data and biometric analysis, using a personalized meal suggestion subsystem of the computing system.

Next at step 1622, the method 1600 involves collecting biometric and biomechanical data via sensors in wearable garments, transmitting data wirelessly to the computing system, and providing real-time feedback, personalized workout recommendations, and progress tracking, using a smart fabrics subsystem of the computing system.

According to an embodiment of the present invention, the step of acquiring biometric data from wearable sensors further comprises establishing a connection between the computing system and a wearable sensor capable of measuring sweat composition, receiving and storing sweat composition data from the wearable sensor in real time and processing the sweat composition data to extract relevant biomarkers, such as sodium, potassium, and glucose levels.

According to an embodiment of the present invention, the step of collecting biometric and biomechanical data via sensors in wearable garments further comprises receiving sensor data from a smart fabric garment worn by the user, analyzing the sensor data to track biometric metrics such as heart rate, respiratory rate, and skin temperature and analyzing the sensor data to track biomechanical metrics such as movement patterns, posture, and muscle activation.

Next at step 1624, the method 1600 involves facilitating communication and data exchange between multiple AI models and enabling holistic health management and personalized engagement based on integrated insights from the AI models, using an AI model interoperability subsystem of the computing system.

Next at step 1626, the method 1600 involves utilizing near-field communication (NFC) to streamline user onboarding, enable the sharing of user profile data between devices, and facilitate personalized marketing and community building, using a contact and data sharing subsystem of the computing system.

Next at step 1628, the method 1600 involves recognizing voice commands, employing AI to generate personalized recommendations, and delivering real-time feedback through natural language responses or auditory cues, using an AI voice interaction and personalized real-time feedback subsystem of the computing system.

According to an embodiment of the present invention, the step of recognizing voice commands further comprises converting captured audio input into text using speech-to-text technology, processing the text using natural language processing techniques to identify user intent and extract relevant information and executing actions or providing feedback based on the interpreted voice command.

According to an embodiment of the present invention, the step of monitoring and recording physical activities further comprises receiving data from wearable devices or user input regarding exercise type, duration, intensity, and distance, calculating calories burned and other relevant metrics based on the received activity data and providing feedback to the user on their activity levels and progress toward fitness goals.

According to an embodiment of the present invention, the step of monitoring and recording user hydration levels further comprises receiving user input regarding water intake throughout the day, calculating and displaying daily, weekly, or monthly hydration levels based on the user input and sending reminders to the user to maintain adequate hydration.

According to an embodiment of the present invention, the step of monitoring and analyzing sleep patterns further comprises receiving and analyzing data from wearable devices or user input regarding sleep duration and quality, analyzing sleep data to identify sleep stages and disturbances and providing feedback and recommendations to the user on improving sleep hygiene.

According to an embodiment of the present invention, the step of providing rewards and incentives further comprises defining fitness goals for the user, such as step count, calories burned, or exercise frequency, tracking the user's progress towards the defined goals and awarding virtual badges, achievements, or points upon reaching specific milestones.

According to an embodiment of the present invention, the step of integrating with social media platforms further comprises enabling users to connect their social media accounts to the computing system, allowing users to share workout summaries, achievements, and progress updates directly to their social media profiles and facilitating social interaction and engagement through comments, likes, and shares on social media posts.

According to an embodiment of the present invention, the step of administering referral incentives and loyalty programs further comprises generating unique referral codes for each user, tracking successful referrals made by users, rewarding users with incentives such as discounts, premium features, or in-app currency for each successful referral and assigning points or rewards to users based on their participation in the loyalty program.

According to an embodiment of the present invention, the step of delivering targeted advertisements and promotions further comprises analyzing user data to identify preferences, demographics, and interests, selecting and displaying relevant advertisements or promotions based on the analyzed user data and tracking user engagement with the advertisements and promotions.

According to an embodiment of the present invention, the step of presenting a catalog of food, beverages, and fitness products further comprises displaying product descriptions, nutritional information, images, and prices, allowing users to browse and search for products by category, brand, or keyword and updating the product catalog dynamically based on inventory and promotional offers.

According to an embodiment of the present invention, the step of providing personalized product recommendations further comprises analyzing user data, such as purchase history, fitness goals, and dietary preferences, generating product recommendations based on the analyzed user data and displaying the personalized recommendations to the user within the computing system.

According to an embodiment of the present invention, the step of facilitating secure shopping, checkout, and order management further comprises allowing users to add products to a virtual shopping cart, providing a secure checkout process for order placement and payment processing, generating order confirmations and tracking numbers and providing options for users to view and manage their order history.

According to an embodiment of the present invention, the step of managing physical beacons in retail locations further comprises configuring and maintaining the operational parameters of beacons, such as signal strength and frequency, monitoring the battery status and connectivity of beacons and updating beacon firmware and software as needed.

According to an embodiment of the present invention, the step of acquiring biometric data from wearable sensors further comprises establishing a connection between the computing system and a wearable sensor capable of measuring sweat composition, receiving and storing sweat composition data from the wearable sensor in real time and processing the sweat composition data to extract relevant biomarkers, such as sodium, potassium, and glucose levels.

According to an embodiment of the present invention, the step of collecting biometric and biomechanical data via sensors in wearable garments further comprises receiving sensor data from a smart fabric garment worn by the user, analyzing the sensor data to track biometric metrics such as heart rate, respiratory rate, and skin temperature and analyzing the sensor data to track biomechanical metrics such as movement patterns, posture, and muscle activation.

According to an embodiment of the present invention, the step of recognizing voice commands further comprises converting captured audio input into text using speech-to-text technology, processing the text using natural language processing techniques to identify user intent and extract relevant information and executing actions or providing feedback based on the interpreted voice command.

The present invention offers numerous advantages over existing fitness related computing systems, revolutionizing the way users approach health and wellness. By combining personalization, holistic tracking, AI-driven insights, community engagement, and sustainable practices, the present invention empowers users to achieve their fitness goals while minimizing environmental impact.

Unlike traditional fitness trackers that focus solely on exercise, this system takes a holistic approach by incorporating features for fasting tracking, hydration monitoring, sleep analysis, and even sustainable lifestyle promotion. This addresses the interconnected nature of health and well-being, leading to more comprehensive and effective results.

The AI model interoperability subsystem enables the system to constantly learn and adapt to the user's evolving needs and preferences. This ensures that recommendations and feedback remain accurate, up-to-date, and effective throughout the user's fitness journey.

The system employs various techniques to enhance user engagement, including gamification (rewards module), community interaction (social media integration and community building modules), and personalized feedback (AI voice interaction subsystem). These features encourage users to stay motivated and committed to their health goals.

The wearable device integration subsystem ensures real-time synchronization of data from fitness trackers and smartwatches. This allows users to monitor their progress and receive timely feedback without any manual input, enhancing convenience and accuracy.

The smart fabrics subsystem incorporates sensors into wearable garments to collect real-time biometric and biomechanical data. This data is used to provide personalized workout recommendations, real-time feedback on form and technique, and detailed progress tracking, leading to safer and more effective workouts.

The integrated marketing capabilities and e-commerce subsystems offer significant revenue generation potential for businesses. Targeted advertising, personalized product recommendations, and loyalty programs can drive user engagement and increase sales.

The sustainable lifestyle promotion subsystem not only reduces the system's environmental impact but also educates and encourages users to adopt eco-friendly habits. This promotes a healthier planet alongside a healthier lifestyle. The personalized meal suggestion subsystem leverages biometric data from wearable sensors to provide tailored recommendations for food and hydration.

The beacon subsystem transforms the retail experience for users by providing personalized promotions, in-store navigation, and seamless payment options. It also provides retailers with valuable data analytics to improve their marketing and sales strategies. The contact and data sharing subsystem simplifies user onboarding and facilitates the sharing of information between users, fostering community building and encouraging social interaction.

Overall, the present invention represents a significant technical advancement in fitness technology. By addressing the limitations of existing applications and incorporating innovative features, the present invention provides a comprehensive, personalized, and sustainable solution that empowers users to achieve their health and fitness goals.

Examples described herein can also be used in various other scenarios and for various purposes. It may be noted that the above-described examples of the present solution are for the purpose of illustration only. Although the solution has been described in conjunction with a specific embodiment thereof, numerous modifications may be possible without materially departing from the instructions and advantages of the subject matter described herein. Other substitutions, modifications, and changes may be made without departing from the spirit of the present solution. All of the features disclosed in this specification (including any accompanying claims, abstract, and drawings), and/or all of the steps of any method or process so disclosed, may be combined in any arrangement, except combinations where at least some of such features and/or steps are mutually exclusive.

The present description has been shown and described with reference to the foregoing examples. It is understood, however, that other forms, details, and examples can be made without departing from the spirit and scope of the present subject matter.

1-21. (canceled)

22. A computing system for integrated fitness tracking and health management, comprising:
a hardware processor; and
a memory coupled to the hardware processor, the memory storing instructions executable by the hardware processor to implement a plurality of software subsystems, the plurality of software subsystems comprising:
a health-management subsystem configured to track one or more user health or fitness parameters;
an adaptive recommendation subsystem configured to generate one or more personalized health or fitness recommendations based at least in part on user-specific data; and
a wearable-device integration subsystem configured to synchronize data from one or more wearable devices and analyze the synchronized data to provide fitness-related output data, wherein the wearable-device integration subsystem includes:
a synchronization module configured to receive and synchronize real-time activity data and biometric information from the one or more wearable devices through a synchronization process that is continuous and automatic without the need for manual data entry;
a data-analysis module configured to process the synchronized data and generate at least one report or visualization for one or more fitness metrics; and
an NFC-based onboarding and data-sharing subsystem configured to use near-field communication to onboard a user or share user data between devices, wherein the NFC-based onboarding and data-sharing subsystem includes:
an NFC communication module configured to establish an NFC connection with a compatible device; and
a data-sharing module configured to be activated in response to detection of the NFC connection to import system data or personalized settings for the user and to perform a bidirectional exchange of data between the computing system and the compatible device.

23. A computing system for integrated fitness tracking and health management, comprising:
a hardware processor; and
a memory coupled to the hardware processor, the memory storing instructions executable by the hardware processor to implement a plurality of software subsystems, the plurality of software subsystems comprising:
a health-management subsystem configured to track one or more user health or fitness parameters;
an adaptive recommendation subsystem configured to generate one or more personalized health or fitness recommendations based at least in part on user-specific data;
a wearable-device integration subsystem configured to synchronize data from one or more wearable devices and analyze the synchronized data to provide fitness-related output data, wherein the wearable-device integration subsystem includes:
a synchronization module configured to receive and synchronize real-time activity data and biometric information from the one or more wearable devices through a synchronization process that is continuous and automatic without the need for manual data entry; and
a data-analysis module configured to process the synchronized data and generate at least one report or visualization for one or more fitness metrics; and
an NFC-based onboarding and data-sharing subsystem configured to use near-field communication to onboard a user and share user profile data between devices, wherein the NFC-based onboarding and data-sharing subsystem includes:
an NFC communication module configured to establish an NFC connection with a compatible device; and
a data-sharing module configured to be activated in response to detection of the NFC connection to import system data or personalized settings to reduce or eliminate manual setup during onboarding and to perform a bidirectional exchange of selected user profile data comprising contact information, profile information, or both between the computing system and the compatible device.

24. A computer-implemented method for integrated fitness tracking and health management, the method being executed by at least one processor and comprising:
tracking, by a health-management subsystem, one or more user health or fitness parameters;
generating, by an adaptive recommendation subsystem, one or more personalized health or fitness recommendations based at least in part on user-specific data;
receiving and synchronizing, by a wearable-device integration subsystem, real-time activity data and biometric information from one or more wearable devices through a synchronization process that is continuous and automatic without manual data entry;
analyzing, by the wearable-device integration subsystem, the synchronized data to provide fitness-related output data;
generating, by a data-analysis module, at least one report or visualization for one or more fitness metrics;
establishing, by an NFC communication module, an NFC connection with a compatible device;
responsive to detecting the NFC connection, importing, by a data-sharing module, system data or personalized settings for a user; and
performing, by the data-sharing module, a bidirectional exchange of data between the computing system and the compatible device.

25. The computer-implemented method of claim 24, wherein the compatible device comprises NFC-enabled product packaging.

26. The computer-implemented method of claim 24, wherein the compatible device comprises a NFC-enabled product.

27. The computer-implemented method of claim 24, wherein the compatible device comprises a product or product packaging having an NFC-enabled component associated with a retail location.

28. The computer-implemented method of claim 24, wherein the compatible device comprises an NFC reader associated with a retail location, and wherein the data-sharing module is configured, responsive to detection of the NFC connection, to import the system data or personalized settings for onboarding of the user and to enable sharing of loyalty program information, reward information, or a discount code associated with a fitness-related food or beverage product.

29. The computer-implemented method of claim 24, wherein the method further comprises managing one or more physical beacons at retail locations, detecting proximity of a user device to the one or more physical beacons, and, responsive to detecting the proximity, using the contact and data sharing subsystem to utilize near-field communication (NFC) to initiate onboarding of the user, exchange selected user profile data, and deliver personalized promotions or navigation based at least in part on user data and a retail location associated with the one or more physical beacons.
